(19) United States
(12) Patent Application Publication
(10) Pub. No.: US 2008/0168984 A1
(43) Pub. Date: Jul. 17, 2008

(54) Inhalation Device

(76) Inventors: Richard David Lintern, Cambridgeshire (GB); Paul Kenneth Rand, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/908,514

(22) PCT Filed: Mar. 16, 2006
(86) PCT No.: PCT/GB2006/000978

§ 371 Date: Sep. 13, 2007

(30) Foreign Application Priority Data
Mar. 17, 2005 (GB) 0505543.9

(51) Int. Cl. A61M 11/00

(52) U.S. Cl. 128/200.23

(57) ABSTRACT

An actuator for an inhaler for delivering medicament by inhalation, comprising: a housing for receiving a canister comprising a body which defines a chamber containing medicament and a valve stem which extends from the body; and a priming mechanism for priming the actuator such as to be actuatable by a user, wherein the priming mechanism comprises a support member which includes a nozzle block for receiving the valve stem of the canister and which is movable relative to the housing between a first, inoperative position in which the canister is inactuatable and a second, primed position in which the canister is actuatable.

### FIELD OF THE INVENTION

The present invention relates to an actuator for an inhaler for administering medicament by inhalation and to an inhaler including the same. The invention is particularly, but not exclusively, concerned with an actuator for a pressurised metered dose inhaler (pMDI).

### BACKGROUND OF THE INVENTION

pMDIs are well known in the art of inhalation devices. It is therefore not necessary to describe the construction and operation of a pMDI other than in bare essentials.

A pMDI comprises a canister and an actuator housing. The housing is generally tubular, although this is not essential, and generally formed of a plastics material, for instance by moulding. The canister comprises an open-ended canister, typically made from a metal such as aluminium. The open end of the canister is sealingly capped by a metering valve assembly. The valve assembly typically includes a hollow dispensing member or valve stem which projects from the outlet or business end of the canister. The dispensing member is mounted for sliding movement relative to the canister between an extended position, to which the dispensing member is biased by a biasing mechanism in the valve assembly, typically a return spring, and a depressed position.

In use, the sealed canister contains a pressurised medicinal aerosol formulation. The formulation comprises the medicament and a fluid propellant, and optionally one or more excipients and/or adjuvants. The medicament is typically in solution or suspension in the formulation. The propellant is typically a CFC-free propellant, suitably a liquid propellant, and may for example be HFA-134a or HFA-227.

Movement of the dispensing member from the extended position to the depressed position results in a metered dose of the aerosol formulation being dispensed from the canister through the dispensing member. Typically, the metering valve assembly is provided with a metering chamber of defined volume. In the extended position of the dispensing member, the content of the canister is placed in fluid communication with the metering chamber through the dispensing member so that the metering chamber is filled with the aerosol formulation. When the dispensing member is depressed, the metering chamber is isolated from the canister inner volume and placed in fluid communication with the external environment through the dispensing member. Thus, the defined volume of the aerosol formulation in the metering chamber is discharged to the external environment via the dispensing member.

Such metering valve assemblies are well known in the art and can be obtained from inter alia Bespak Plc (King's Lynn, Norfolk, United Kingdom) and Valois S. A. S. (Le Neubourg, France).

The housing typically comprises an internal passageway having an open end. The canister is slidable into the internal passageway through the open end with the canister being inserted valve assembly first into the internal passageway. A stem block, which receives the dispensing member of the canister when the canister is received in the housing in a “rest position”, has a passageway with an inlet end for receiving the dispensing member and an outlet end, which faces a dispensing outlet of the housing, typically a mouthpiece or a nasal nozzle. The stem block holds the dispensing member stationary whereby depression of the canister to its rest position further into the housing to an “actuated position” causes the dispensing member to be displaced from the extended position to the depressed position relative to the canister. A metered dose of the aerosol formulation will thereby be dispensed out of the dispensing outlet of the housing via the internal passageway of the stem block.

In use, a patient in need of a metered dose of the medicinal aerosol formulation concurrently inhales on the dispensing outlet and depresses the canister from the rest position to the actuated position. The inspiratory airflow produced by the patient entrains the metered dose of the medicinal aerosol formulation into the patient's respiratory tract. So, a pMDI of the type described above is a breath-coordinated inhaler.

Inhalers are commonly provided with a dust cap that covers the dispensing outlet when the inhaler is not in use. The dust cap, when applied, prevents foreign material from entering the housing. This prevents the user from inhaling dust or lint, for example, that might otherwise accumulate in the housing. This is of particular importance where the user suffers from asthma or other respiratory conditions, in which the inhalation of foreign material may cause severe irritation.

Developments to pMDIs have included the provision of actuation indicators or dose counters therefor. Such a dose counter is described in PCT Patent Application Nos. WO-A-9856444 and WO-A-2004/001664 to Glaxo Group Limited. The dose counter is fixably secured on the valve assembly end of the canister and includes a display which denotes the number of metered doses of the medicament formulation dispensed from, or remaining in, the canister. The display of the dose counter is visible to the patient through a window provided in the housing. The display may be presented by a plurality of indicator wheels rotatably mounted on a common axle, each wheel having numerals displayed in series around the circumference.

Many actuators have been developed with a view to facilitating the delivery of medicament, examples of which are disclosed in U.S. Pat. No. 3,272,391, U.S. Pat. No. 3,272,392, U.S. Pat. No. 4,678,106, U.S. Pat. No. 5,899,365, U.S. Pat. No. 6,237,812 and WO-A-99/49917.

It is an aim of the present invention to provide an improved actuator for an inhaler for administering medicament by inhalation and an inhaler including the same.

### SUMMARY OF THE INVENTION

In one aspect the present invention provides an actuator for an inhaler for delivering medicament by inhalation, comprising: a housing for receiving a canister comprising a body which defines a chamber containing medicament and a valve stem which extends from the body; and a priming mechanism for priming the actuator such as to be actuatable by a user, wherein the priming mechanism comprises a support member which includes a nozzle block for receiving the valve stem of the canister and is movable relative to the housing between a first, inoperative position in which the canister is inactuatable and a second, primed position in which the canister is actuatable.

Preferably, the priming mechanism further comprises a drive member for moving the support member between the inoperative and primed positions.

More preferably, the support member includes a cam surface and the drive member comprises a cam element which includes a cam surface which engages the cam surface of the support member, such that, on movement of the drive member between a first, inoperative position and a second, primed position, the support member is moved between the inoperative and primed positions.

Yet more preferably, the support member includes a cam bore which defines the cam surface of the support member and the cam element is a rotatable element which is disposed within the cam bore and rotatable between the inoperative and primed positions.

In one embodiment the housing includes an outlet through which the user in use inhales, and further comprising: a cap which, in a closed position, encloses the outlet, wherein the cap is operably connected to the drive member, such that movement of the cap from the closed position to an open position drives the drive member between the inoperative and primed positions.

Preferably, the outlet is a mouthpiece.

Preferably, the cap is pivotally coupled to the drive member and includes an engagement lug which engages the drive member on pivoting the cap from the closed position, such that the cap becomes a substantially rigid extension of the drive member.

Preferably, the housing includes a stop member which prevents actuation of the canister until the support member is in the primed position.

Preferably, the actuator further comprises: an actuating mechanism for actuating the canister.

More preferably, the actuating mechanism comprises at least one actuating member which is movably disposed relative to the housing between a first, inner position and a second, outer deployed position in which the at least one actuating member is presented for actuation by the user.

In one embodiment the actuating mechanism is operably coupled to the priming mechanism, such that the at least one actuating member is moved between the inner and deployed positions on movement of the support member between the inoperative and primed positions.

Preferably, the actuating mechanism further comprises a loading member which is locatable on the body of the canister and includes a loading section which is coupled to the at least one actuating member, such that the at least one actuating member is movable in response to movement of the canister and the canister is movable in response to depression of the at least one actuating member when the support member is in the primed position.

In another embodiment the actuating mechanism includes a biasing element which normally biases the at least one actuating member to the outer, deployed position.

Preferably, the actuating mechanism further comprises a loading member which is locatable on the body of the canister and includes a loading section which is engageable with the at least one actuating member, such that the canister is movable in response to depression of the at least one actuating member when the support member is in the primed position.

Preferably, the loading member is a cap element which comprises a sleeve which is adapted to fit about an outer peripheral wall of the body of the canister, an end section at one end of the sleeve which is adapted to engage a base of the body of the canister, and the loading section at the other end of the sleeve.

Preferably, the loading section comprises an outwardly-extending flange.

Preferably, the at least one actuating member is pivotally coupled to the housing and comprises a first, gripping element which extends from the pivot along a length of the housing and is configured to be gripped and depressed by the user in actuating the canister, and a second, loading element which extends inwardly from the pivot and engages the loading section of the loading member.

Preferably, the actuating mechanism comprises first and second actuating members disposed in oppositely-directed relation.

The present invention also extends to an inhaler comprising the above-described actuator and a canister containing medicament.

Other aspects and features of the invention are set forth in the appended claims and the exemplary embodiments which will now be described with reference to the accompanying Figures of drawings.

### BRIEF DESCRIPTION OF THE DRAWINGS

FIGS. 1(a) and (b) illustrate part-sectional front and side views of an inhaler in accordance with a first embodiment of the present invention, where illustrated in the closed, inoperative configuration;

FIGS. 2(a) and (b) illustrate part-sectional front and side views of the inhaler of FIG. 1, where illustrated with the mouthpiece cap partially removed from the mouthpiece;

FIGS. 3(a) and (b) illustrate part-sectional front and side views of the inhaler of FIG. 1, where illustrated in an intermediate configuration;

FIGS. 4(a) and (b) illustrate part-sectional front and side views of the inhaler of FIG. 1, illustrated in the operative, primed configuration, ready for actuation by a user;

FIGS. 5(a) and (b) illustrate part-sectional front and side views of the inhaler of FIG. 1, illustrated in the actuated configuration, following actuation by the user;

FIGS. 6(a) and (b) illustrate part-sectional front and side views of an inhaler in accordance with a second embodiment of the present invention, where illustrated in the closed, inoperative configuration;

FIGS. 7(a) and (b) illustrate part-sectional front and side views of the inhaler of FIG. 6, where illustrated with the mouthpiece cap partially removed from the mouthpiece;

FIGS. 8(a) and (b) illustrate part-sectional front and side views of the inhaler of FIG. 6, where illustrated in an intermediate configuration;

FIGS. 9(a) and (b) illustrate part-sectional front and side views of the inhaler of FIG. 6, illustrated in the operative, primed configuration, ready for actuation by a user; and

FIGS. 10(a) and (b) illustrate part-sectional front and side views of the inhaler of FIG. 6, illustrated in the actuated configuration, following actuation by the user.

### DETAILED DESCRIPTION OF EXEMPLARY EMBODIMENTS

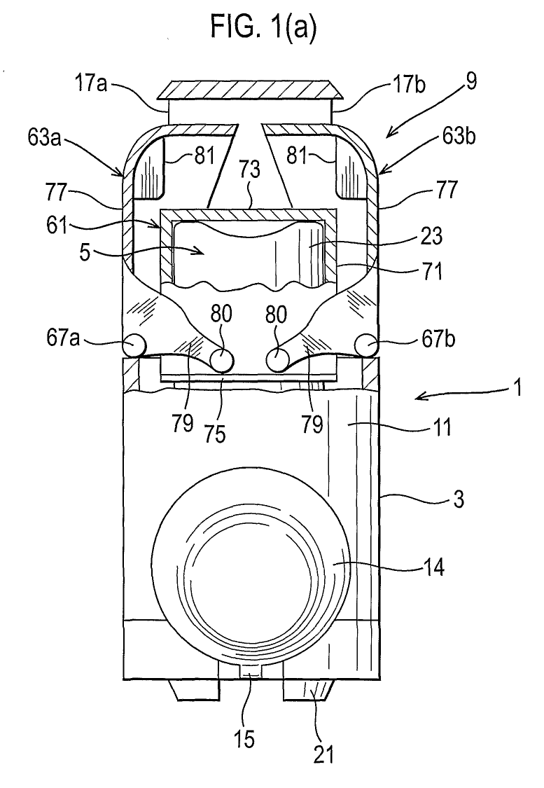

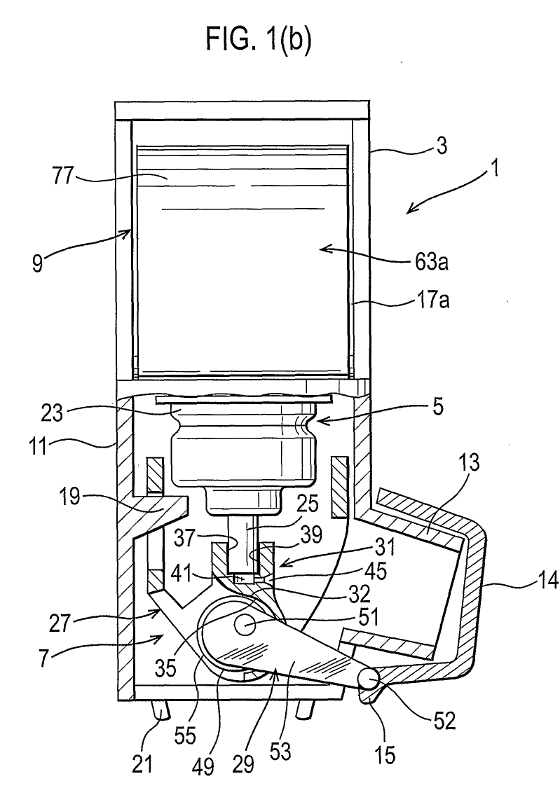

FIGS. 1 to 5 illustrate a hand-held, hand-operable inhaler of the pMDI type in accordance with a first embodiment of the present invention.

The inhaler comprises an actuator 1 which comprises a main body 3, an aerosol canister 5 of the previously described standard type which is fitted in the main body 3 and contains medicament to be delivered on actuation of the inhaler, a priming mechanism 7 for priming the inhaler, and an actuating mechanism 9 which is operable by a user to actuate the inhaler.

The main body 3 comprises a housing 11 in which the canister 5 is in use fitted, and a mouthpiece 13, in this embodiment a tubular element, which is in fluid communication with a lower end of the housing 11 and in use is gripped in the lips of the user. The mouthpiece 13 could instead be configured as a nasal nozzle.

The inhaler further comprises a mouthpiece cap 14 which provides for closure of the mouthpiece 13, and, as will be described in more detail hereinbelow, is operably coupled to the priming mechanism 7. In this embodiment the mouthpiece cap 14 includes an engagement lug 15 at a lower edge thereof, the purpose of which will become apparent hereinbelow.

The housing 11 includes first and second lateral apertures 17a, b at an upper end thereof, which are disposed in opposed relation to lateral sides of the mouthpiece 13 and receive actuating members 63a, b of the actuation mechanism 9, as will be described in more detail hereinbelow.

In this embodiment the housing 11 further includes a stop member 19, here an internal projection, which acts to prevent actuation of the canister 5 prior to configuration of the inhaler in the primed, operative configuration, as will be described in more detail hereinbelow.

In this embodiment the housing 11 further includes a catch member 21, here comprising a pair of catch elements, which is disposed on a lower surface of the housing 11 adjacent the mouthpiece 13 and acts to retain the mouthpiece cap 14 in an open position, which corresponds to the operative, primed configuration of the inhaler, as will be described in more detail hereinbelow.

The canister 5 in this embodiment is of standard type, as outlined supra, and comprises a canister body 23 which defines a chamber containing a medicament in a CFC-free propellant under pressure, for example an HFA propellant, a valve stem 25 which extends from one end of the canister body 23 and an internal metering valve (not illustrated) which is normally biased to a closed position by an internal valve spring (not illustrated) and opened to deliver a metered dose of medicament from the canister 5 when the valve stem 25 is depressed.

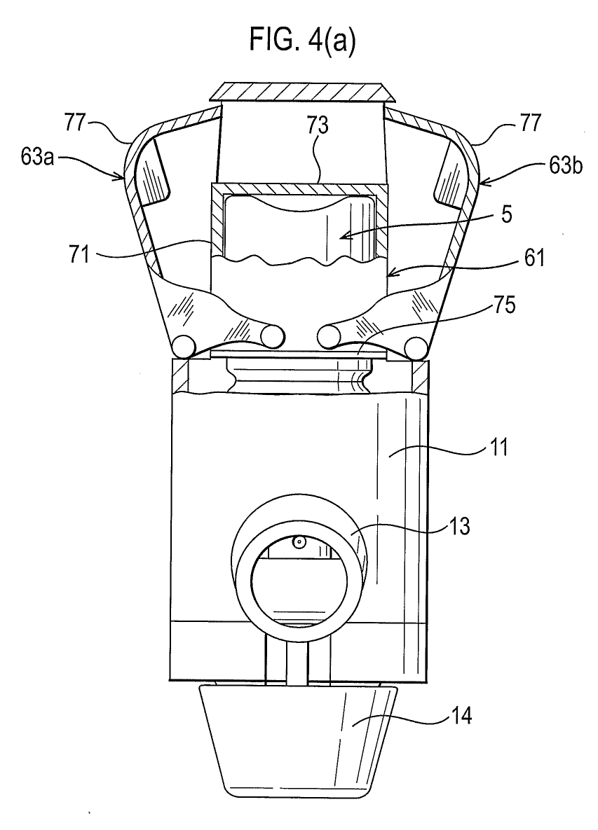

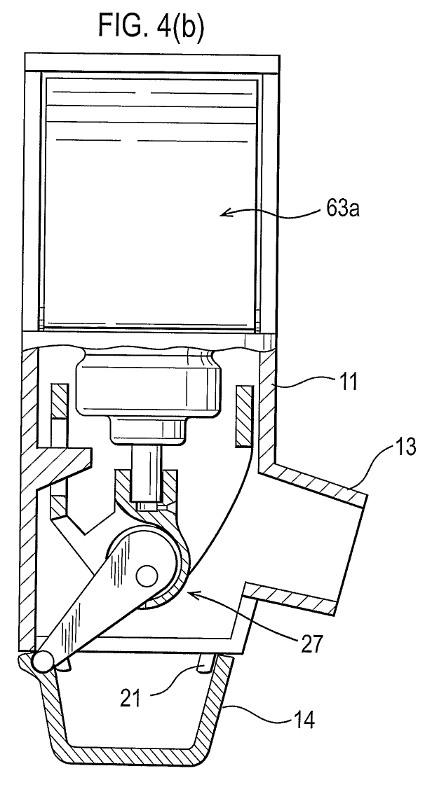

The priming mechanism 7 comprises a support member 27 to which the canister 5 is mounted, which is movable between a first, lower inoperative position, as illustrated in FIG. 1(b), and a second, raised primed position, as illustrated in FIG. 4(b), and a drive member 29 which is operable to drive the support member 27 between the inoperative and primed positions.

In this embodiment the support member 27 includes a nozzle block 31 for receiving the valve stem 25 of the canister 5, and a laterally-directed cam bore 32 which extends orthogonally to the longitudinal axis of the housing 11 and defines a cam surface 35 which is engaged by the drive member 29 to drive the support member 27 between the inoperative and primed positions.

The nozzle block 25 includes a tubular bore 37 for receiving the valve stem 25 of the canister 5, which in this embodiment is co-axial with the longitudinal axis of the housing 11. The tubular bore 37 is open at one, the upper, end thereof and includes an upper section 39 which has an internal dimension which is substantially the same as the outer dimension of the valve stem 25 and a lower section 41 which has a smaller dimension, which sections 39, 41 together define an annular seat for the distal end of the valve stem 25. The tubular bore 37 further includes a laterally-directed spray orifice 45 in the lower section 41 thereof which is configured to direct a spray of the medicament formulation dispensed from the valve stem into and through the mouthpiece 13.

In this embodiment the drive member 29 comprises a cam element 49 which is mounted about a pivot 51 and extends through the cam bore 32 of the support member 27, and a drive arm 53 which is pivotally connected to the mouthpiece cap 14 about a pivot 52 at one, the distal, end and fixedly connected to the cam element 49 at the other end, such as to be operative to move the same between an inoperative position, as illustrated in FIG. 1(b) and a primed, operative position, as illustrated in FIG. 4(b). The cam element 49, in this embodiment a cylindrical rod which is eccentrically mounted about the pivot 51, defines a cam surface 55 which engages the cam surface 35 of the cam bore 32 of the support member 27, such as to cause the support member 27 to adopt the lower, inoperative position with the drive arm 53 in the inoperative position and the raised, primed position with the drive arm 53 in the primed, operative position.

The actuating mechanism 9 comprises a loading member 61 which is fitted over the base of the canister body 23 of the canister 5, and first and second actuating members 63a, b which are disposed at the respective ones of the lateral apertures 17a, b in the housing 11 and pivoted about respective pivots 67a, b to the housing 11 between a first, inner configuration, as illustrated in FIG. 1(a), and a second, outer deployed configuration, as illustrated in FIG. 4(a), such as to provide for actuation of the canister 5 by engagement with the loading member 61, when the priming mechanism 7 is in the primed configuration.

In this embodiment the loading member 61 is a cap element which comprises a sleeve 71, here a tubular sleeve, which is a close fit with the outer peripheral wall of the canister body 23 of the canister 5, an end section 73 at one, the upper, end of the sleeve 71, here which spans the sleeve 71, which engages the base of the canister body 23 of the canister 5, and a loading section 75, here first and second lateral flanges or an annular flange, at the other, lower end of the sleeve 71, which is engaged by the actuating members 63a, b to load the canister 5, as will be described in more detail hereinbelow.

In this embodiment the actuating members 63a, b each comprise a first, gripping arm 77 which extends substantially upwardly from the respective pivot 67a, b and is configured to be gripped and depressed by the user in actuating the inhaler (e.g. with opposing digits of a user's hand), and a pair of second, loading arms 79 (only one shown) which extend inwardly from the respective pivot 67a, b, in a direction substantially orthogonal to the gripping arm 77 so as to straddle the loading member 61, thus defining substantially an inwardly-directed L shape, and connected at point 80 on the loading section 75 of the loading member 61, such that the loading member 61 and the actuating members 63a, b move together.

In this embodiment the actuating members 63a, b each further include a stop member 81, here an internal projection, which acts to prevent the upward movement, and hence escape, of the canister 5 from the support member 27 when the actuating members 63a, b are in the inner configuration.

Operation of the actuator will now be described hereinbelow.

The user first takes the actuator in the closed, inoperative configuration, as illustrated in FIGS. 1(a) and (b), in one hand.

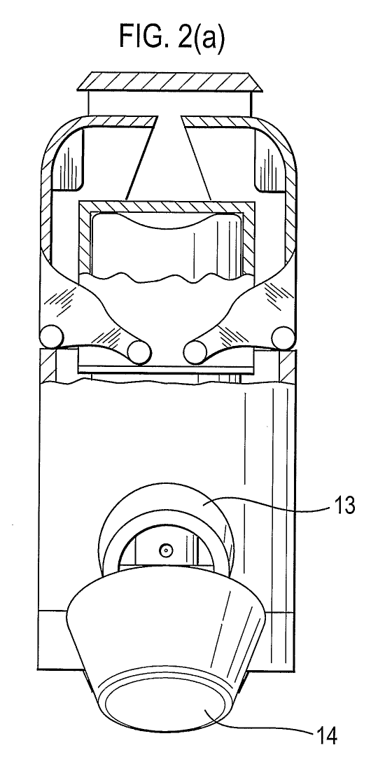

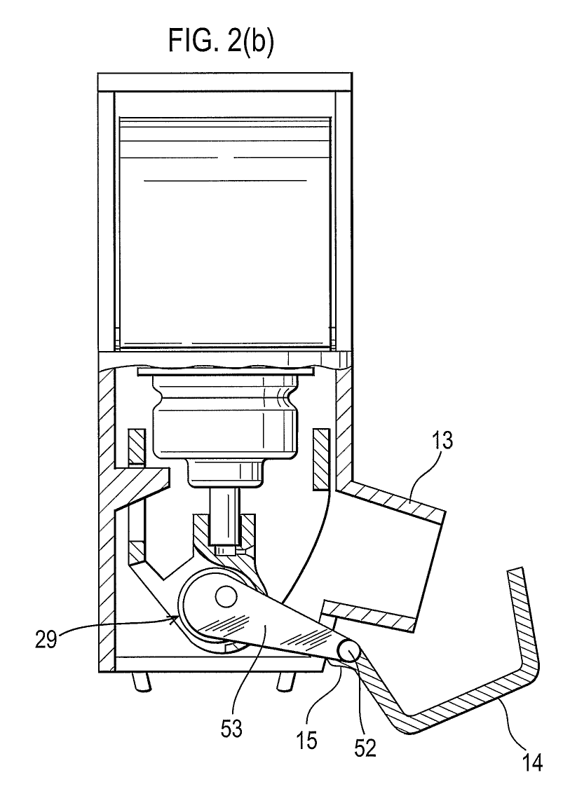

The user removes the mouthpiece cap 14, in this embodiment by rotating or “flipping” the same downwards to reveal the mouthpiece 13, as illustrated in FIGS. 2(a) and (b). In this position, the engagement lug 15 on the mouthpiece cap 14 engages the drive arm 53 of the drive member 29.

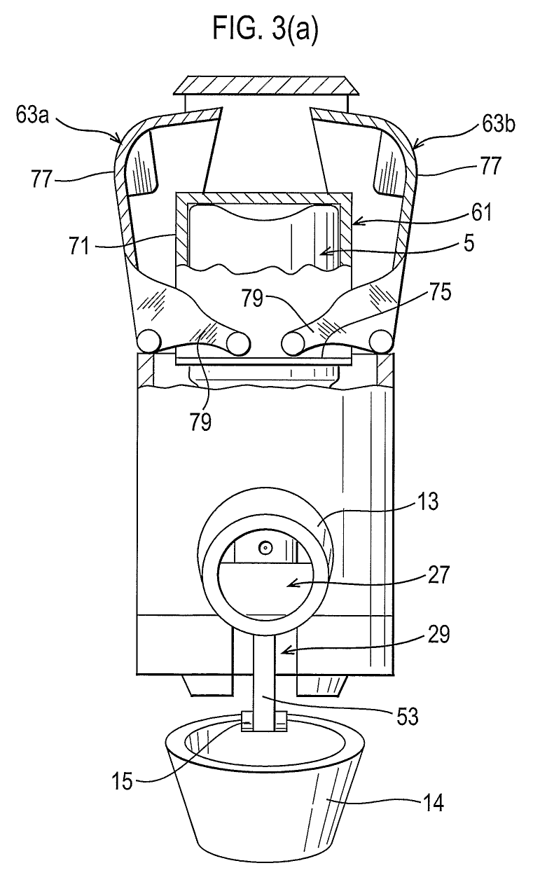

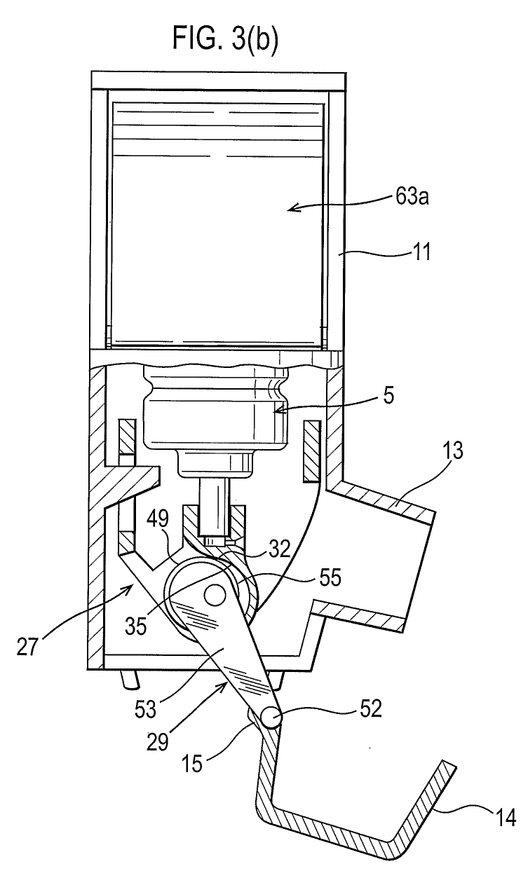

The user then continues to rotate the mouthpiece cap 14 downwards, which action, through engagement of the engagement lug 15 of the mouthpiece cap 14 and the drive arm 53 of the drive member 29, causes rotation of the drive member 29, as illustrated in FIGS. 3(a) and (b). This operation of the drive member 29, through engagement of the cam surface 55 of the cam element 49 of the drive member 29 and the cam surface 35 of the cam bore 32 of the support member 27, causes the support member 27 to be raised from the lower, inoperative position.

This upward movement of the support member 27 causes the canister 5 to be moved upwards, which movement raises the loading member 61 and causes the gripping arms 77 of the actuating members 63a, b, through engagement of the loading section 75 of the loading member 61 and the loading arms 79 of the actuating members 63a, b, to be deployed by the outward rotation thereof.

On completion of the rotation of the mouthpiece cap 14, the mouthpiece cap 14 is engaged by the catch member 21 on the lower surface of the housing 11 to hold the same in the open, operative position, the support member 27 is fully raised to the operative, primed position and the gripping arms 77 of the actuating members 63a, b are fully outwardly deployed, as illustrated in FIGS. 4(a) and (b).

The user then takes the mouthpiece 13 in his/her lips, and, in co-ordination with an inhalation breath, actuates the inhaler by depressing the gripping arms 77 of the actuating members 63a, b.

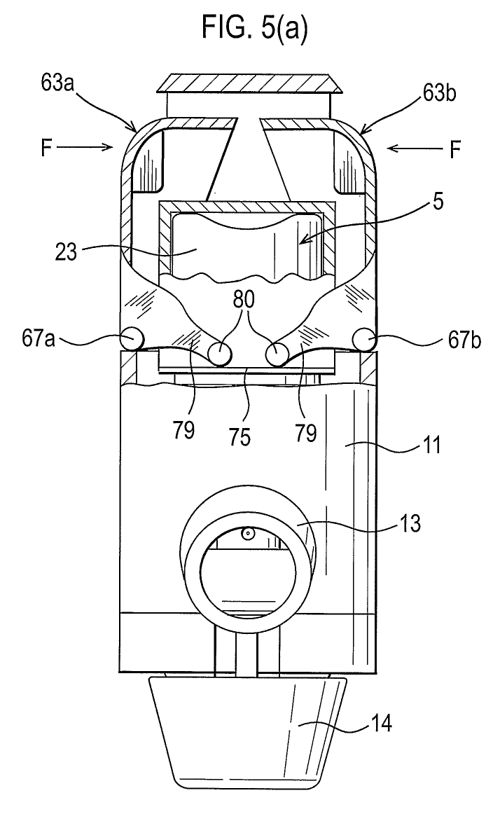

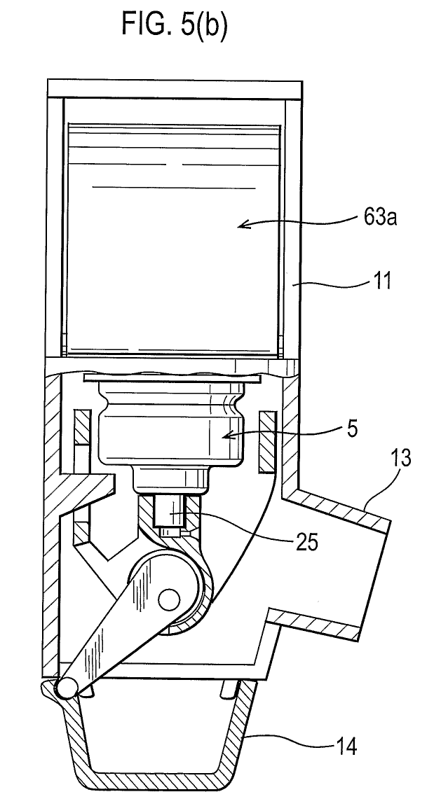

As illustrated in FIGS. 5(a) and (b), depression of the gripping arms 77 of the actuating members 63a, b causes the inward rotation of the actuating members 63a, b, such that the loading arms 79 of the actuating members 63a, b drive the loading section 75 of the loading member 61, and hence the loading member 61, downwardly, which downward movement of the loading member 61 drives the canister body 23 of the canister 5 downwardly in relation to the stationary valve stem 25 of the canister 5, the position of which is fixed by the support member 27 which is held in the raised, primed position, thus actuating the canister 5 to deliver a spray of the medicament formulation into and through the mouthpiece 13.

Following actuation, the inhaler is removed from the mouth, and the actuating members 63a, b are released. On releasing the actuating members 63a, b, the inhaler is returned by the return spring in the metering valve assembly of the canister 5 to the primed configuration, as illustrated in FIGS. 4(a) and 4(b). The mouthpiece cap 14 is then returned to the closed position, as illustrated in FIGS. 1(a) and (b), ready for subsequent actuation.

As will be appreciated, the re-closing of the cap 14 causes downward movement of the support member 27, which in turn causes downward movement of the canister 5 due to an interference fit of the valve stem 25 in the nozzle block 31, which in turn pulls down the loading member 61 fitted about the canister 5, which in turn causes the actuating members 63a, b to be returned to the inner configuration due to their connection to the loading member 61.

It will also be appreciated that when the mouthpiece cap 14 is in the closed position, shown in FIGS. 1(a) and (b), it is not possible for the user to operate the inhaler. This is because the projection 19 prevents the canister body 23 being displaced downwardly enough relative to the stationary valve stem 25 to cause the metering valve assembly to open for discharge therefrom. This, of course, is not the case when the support member 27, and the inhaler parts supported thereby, is moved upwardly relative to the projection 19 by movement of the mouthpiece cap 14 to its open, operative position, shown in FIGS. 4(a) and (b).

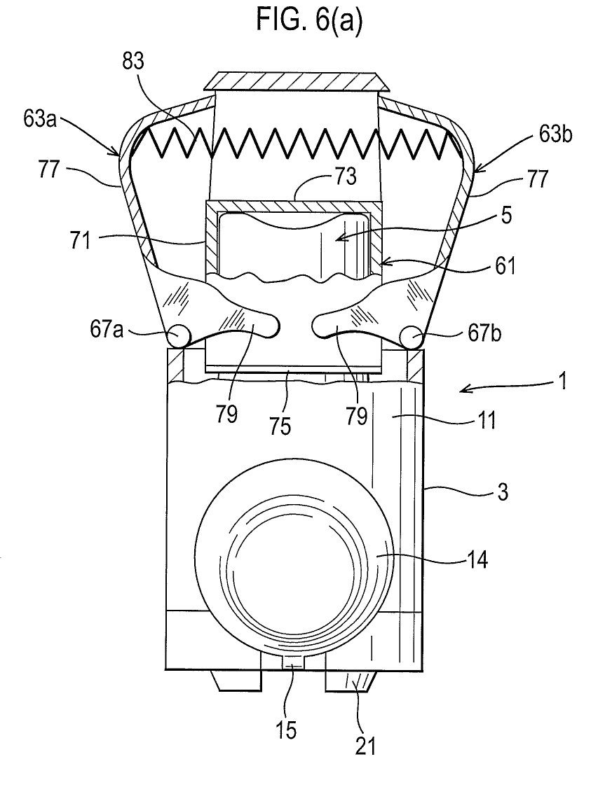

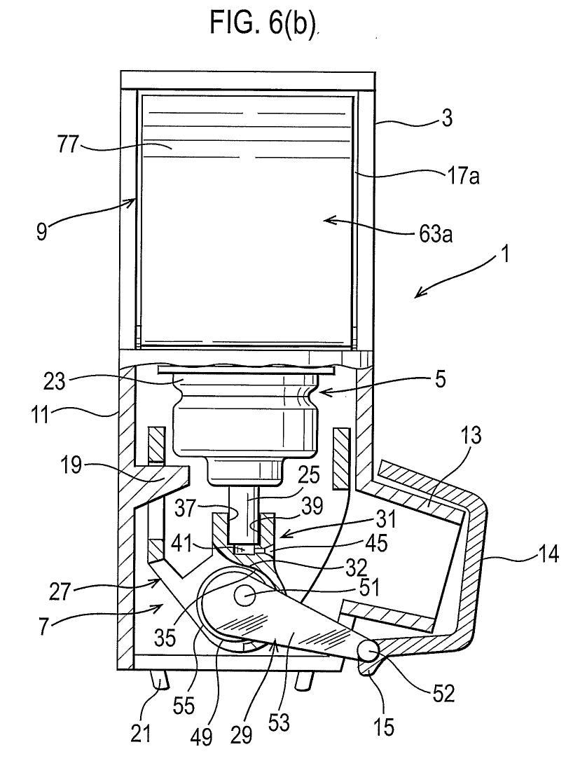

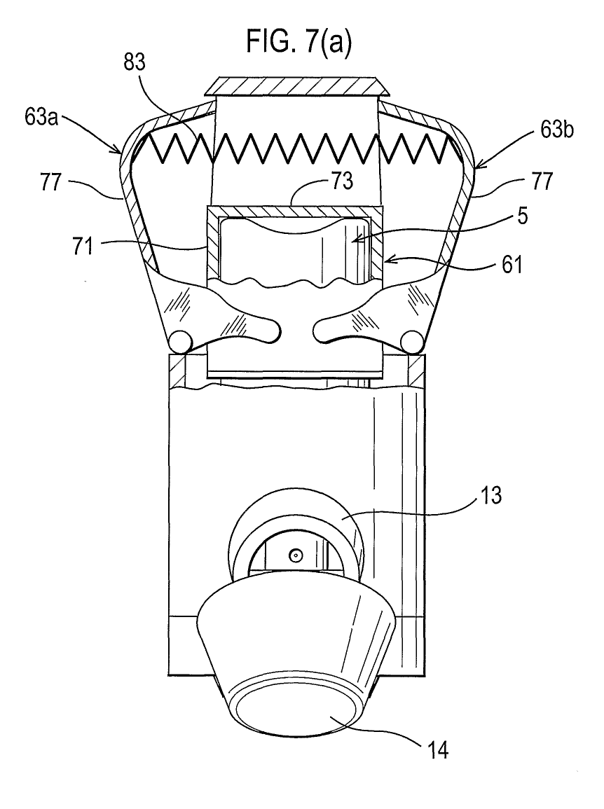

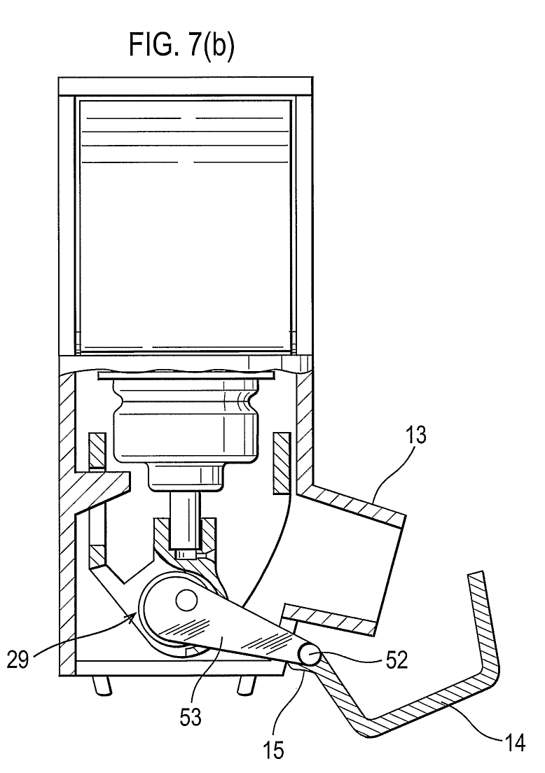

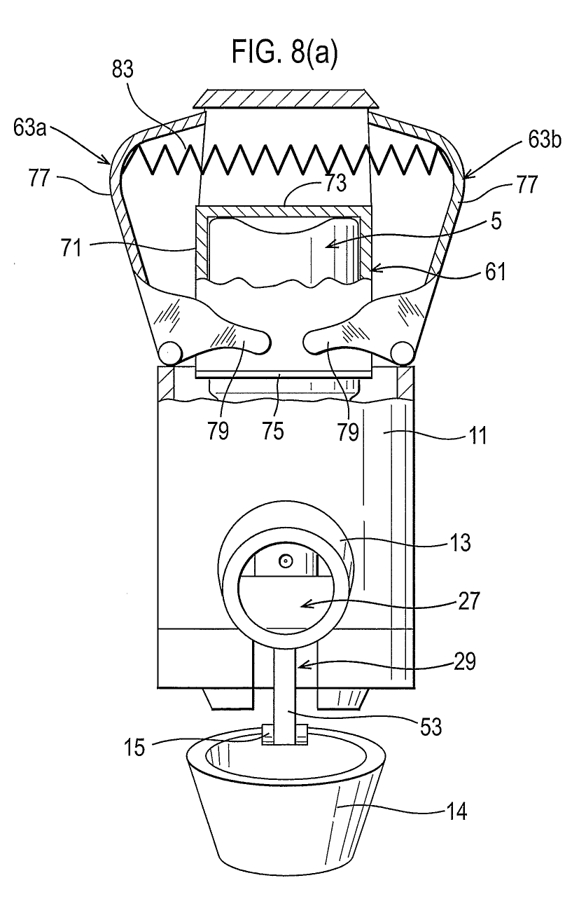

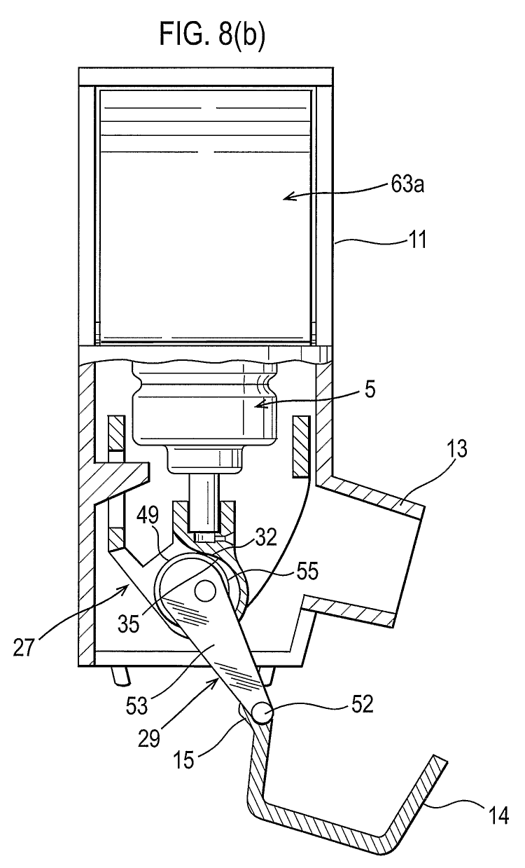

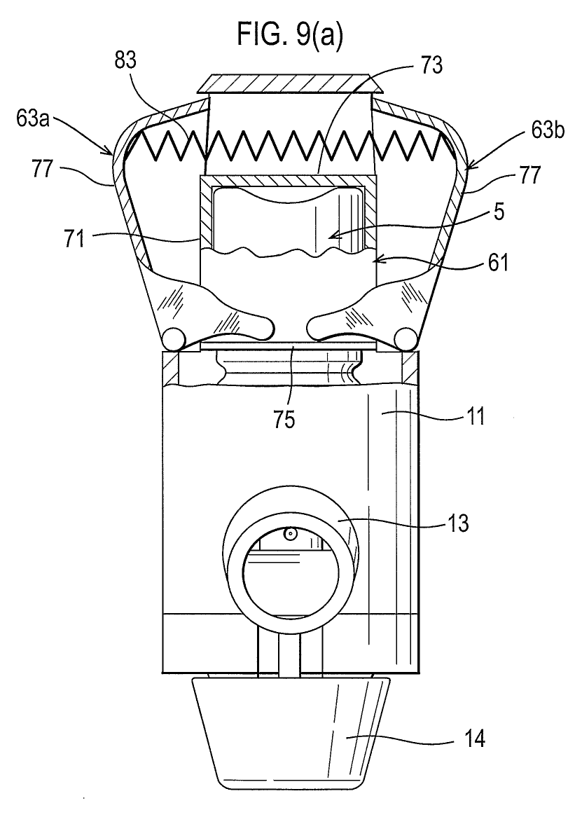

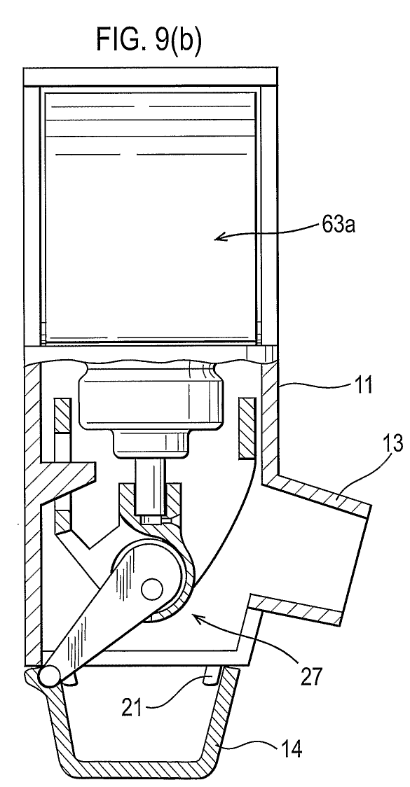

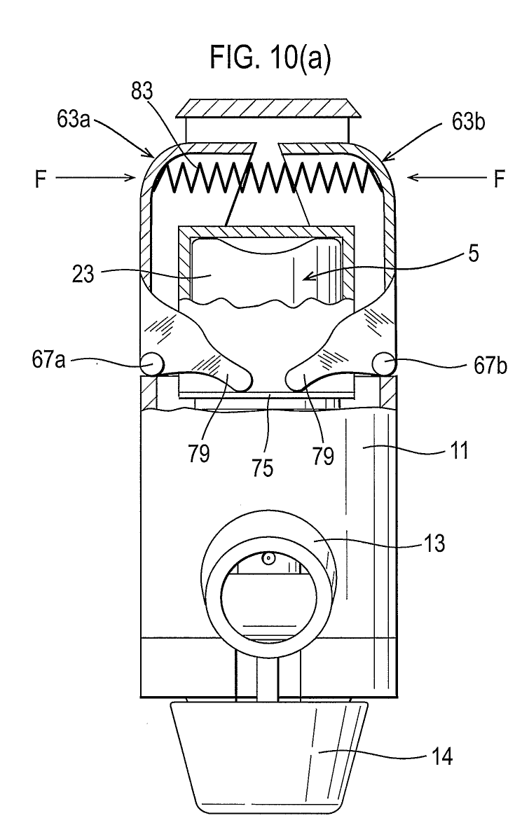

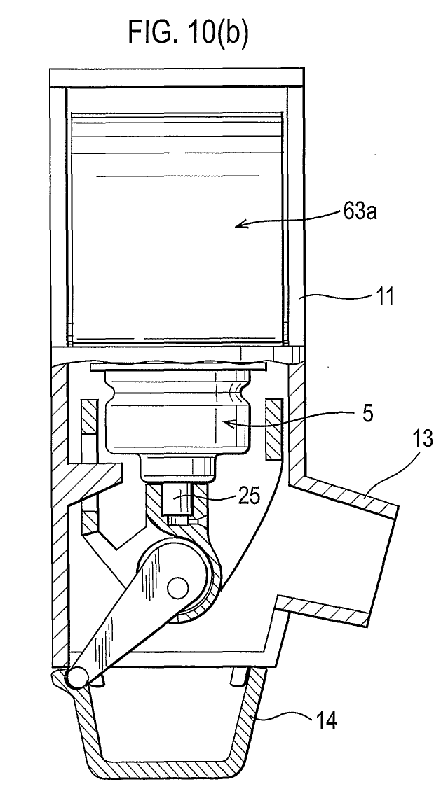

FIGS. 6 to 10 illustrate a hand-held, hand-operable inhaler of the pMDI type in accordance with a second embodiment of the present invention.

The inhaler of this embodiment is very similar to the inhaler of the above-described embodiment, and thus, in order to avoid unnecessary duplication of description, only the differences will be described in detail, with like parts being designated by like reference signs.

The inhaler of this embodiment differs from that of the first-described embodiment in the construction of the actuating mechanism 9. In this embodiment the actuating members 63a, b are not coupled to the loading member 61 and the actuating mechanism 9 includes a biasing element 83 which acts normally to bias the gripping arms 77 of the actuating members 63a, b outwardly to the outer deployed positions. With this configuration, the actuating members 63a, b always normally adopt the outer deployed positions, as illustrated in FIGS. 6 to 9. Whilst this configuration allows the user to depress the gripping arms 77 of the actuating members 63a, b at any time, the actuator still remains inoperative until the priming mechanism 7 is in the primed configuration and the loading member 61 is raised to the primed position, as, until the loading member 61 reaches that position, the depression of the actuating members 63a, b, through engagement of the loading arms 79 thereof and the loading member 61, does not provide for sufficient movement of the canister 5 to effect actuation due to the positioning and blocking action of the projection 19, as described previously with reference to the first embodiment.

The inhaler of this embodiment further differs from that of the first-described embodiment in that the actuating members 63a, b each do not include the stop member 81.

Operation of the inhaler of this embodiment is the same as for the above-described embodiment.

It will be appreciated that the actuating mechanisms 9 in the illustrated embodiments of the invention provide a mechanical advantage. That is to say, the manual force required to be applied by the user to operate the inhaler (by overcoming the return force of the valve return spring) is less than would otherwise be the case, such as in operation of a standard pMDI where the user has to push down on the base of the canister 5 against the return force of the valve return spring.

Preferably, all of the parts of the actuator 1 of the exemplary embodiments are made from a plastics material, for example by a moulding process.

In a modification of the illustrated embodiments, the loading member 61 takes the form of an accessory which is fixedly connected to the head end of the canister 5 and which provides the loading section 75 for the actuating members 63a, b to act on to move the canister 5 downwardly when the inhaler is in the primed configuration. As an example, the accessory may take the form of a dose counter as described in WO-A-9856444 and WO-A-2004/001664, the entire contents of which are hereby incorporated herein by reference. The housing of such a dose counter may be adapted to present the loading section.

In an alternative modification of the illustrated embodiments, not shown, the loading section 75 for the actuating members 63a, b may be presented by a surface of the canister 5.

Finally, it will be understood that the present invention has been described in its exemplary embodiments and can be modified in many different ways without departing from the scope of the invention as defined by the appended claims.

Also, as regards the provision of reference signs in the appended claims, it is to be understood that reference signs are provided only for illustrative purposes and are not intended to confer any limitation to the claimed invention.

What is claimed is:

1. An actuator for an inhaler for delivering medicament by inhalation, comprising:
a housing for receiving a canister comprising a body which defines a chamber containing medicament and a valve stem which extends from the body; and
a priming mechanism for priming the actuator such as to be actuatable by a user, wherein the priming mechanism comprises a support member which includes a nozzle block for receiving the valve stem of the canister which is movable relative to the housing between a first, inoperative position in which the canister is inactuatable and a second, primed position in which the canister is actuatable.

2. The actuator of claim 1, wherein the priming mechanism further comprises a drive member for moving the support member between the inoperative and primed positions.

3. The actuator of claim 2, wherein the support member includes a cam surface and the drive member comprises a cam element which includes a cam surface W which engages the cam surface of the support member, such that, on movement of the drive member between a first, inoperative position and a second, primed position, the support member is moved between the inoperative and primed positions.

4. The actuator of claim 3, wherein the support member includes a cam bore which defines the cam surface of the support member and the cam element is a rotatable element which is disposed within the cam bore and rotatable between the inoperative and primed positions.

5. The actuator of claim 1, wherein the housing includes an outlet through which the user in use inhales, and further comprising:
a cap which is movable between a closed position, in which the cap encloses the outlet, and an open position, in which the cap does not enclose the outlet, wherein movement of the cap between its closed and open positions moves the support member between its inoperative and primed positions.

6. The actuator of claim 5, wherein the outlet is a mouthpiece.

7. The actuator of claim 1, wherein the housing includes an outlet through which the user in use inhales and further comprising:
a cap which is movable between a closed position in which the cap encloses the outlet and an open position, in which the cap does not enclose the outlet
wherein movement of the cap between its closed and open positions moves the support member between its inoperative and primed positions
wherein the outlet is a mouthpiece and
wherein the cap is pivotally coupled to the drive member and optionally includes an engagement lug which engages the drive member on pivoting the cap from the closed position, such that the cap becomes a substantially rigid extension of the drive member.

8. The actuator of claim 1, wherein the housing includes a stop member which prevents actuation of the canister until the support member is in the primed position.

9. The actuator of claim 1 further comprising
an actuating mechanism for actuating the canister.

10. The actuator of claim 9, wherein the actuating mechanism comprises at least one actuating member (63a, b) which is movably disposed relative to the housing between a first, inner position and a second, outer deployed position in which the at least one actuating member is presented for actuation by the user.

11. The actuator of claim 10, wherein the actuating mechanism is operably coupled to the priming mechanism, such that the at least one actuating member is moved between the inner and deployed positions on movement of the support member between the inoperative and primed positions.

12. The actuator of claim 10, wherein the actuating mechanism includes a biasing element which normally biases the at least one actuating member to the outer, deployed position.

13. The actuator of claim 10, wherein the actuating mechanism further comprises a loading member which is locatable on the body the canister and includes a loading section which is coupled to the at least one actuating member, such that the at least one actuating member is movable in response to movement of the canister and the canister is movable in response to depression of the at least one actuating member when the support member is in the primed position.

14. The actuator of claim 10, wherein the actuating mechanism further comprises a loading member which is locatable on the body of the canister and includes a loading section which is engageable with the at least one actuating member, such that the canister is movable in response to depression of the at least one actuating member when the support member is in the primed position.

15. The actuator of claim 13, wherein the loading member is a cap element which comprises a sleeve which is adapted to fit about an outer peripheral wall of the body of the canister, an end section at one end of the sleeve which is adapted to engage a base of the body of the canister, and the loading section at the other end of the sleeve.

16. The actuator of claim 13, wherein the loading section comprises an outwardly-extending flange.

17. The actuator of claim 13, wherein the at least one actuating member is pivotally coupled to the housing and comprises a first, gripping element which extends from the pivot along a length of the housing and is configured to be gripped and depressed by the user in actuating the canister, and a second, loading element which extends inwardly from the pivot and engages the loading section of the loading member.

18. The actuator of claim 9, wherein the actuating mechanism comprises first and second actuating members disposed in oppositely-directed relation.

19-21. (canceled)
